(19) United States
(12) Patent Application Publication
(10) Pub. No.: US 2025/0295853 A1
(43) Pub. Date: Sep. 25, 2025

(54) SUBARACHNOID FLUID MANAGEMENT METHOD AND SYSTEM WITH VARYING RATES

(71) Applicant: EnClear Therapies, Inc., Newburyport, MA (US)

(72) Inventors: Gianna N. Riccardi, South Berwick, ME (US); William X. Siopes, Jr., Lowell, MA (US); Marcie Glicksman, Salem, MA (US); Anthony DePasqua, Newburyport, MA (US); Kevin Kalish, Newburyport, MA (US); Joshua G. Vose, Seattle, WA (US); Rajan Patel, Somerville, MA (US)

(21) Appl. No.: 19/231,172

(22) Filed: Jun. 6, 2025

Related U.S. Application Data
(63) Continuation of application No. 17/489,620, filed on Sep. 29, 2021, now Pat. No. 12,337,140.
(60) Provisional application No. 63/084,996, filed on Sep. 29, 2020.
(60) Provisional application No. 63/117,975, filed on Nov. 24, 2020.

(51) Int. Cl. A61M 5/168; A61M 5/142; A61M 5/145

(52) U.S. Cl. CPC A61M 5/16827; A61M 5/145; A61M 5/16886; A61M 2005/14208; A61M 2202/0464; A61M 2205/3344; A61M 2205/3389; A61M 2210/0693

(57) ABSTRACT

A CSF management method for use with a patient forms a closed loop CSF circuit between two points on the patient's body. The CSF circuit has a therapeutic inlet to receive a therapeutic material (e.g., a drug), and a pump having a pump outlet to direct CSF along the CSF circuit. The method controls the pump to direct CSF from the pump outlet at a CSF rate that is different from the natural flow rate (i.e., the natural CSF flow rate). The therapeutic material is added to the CSF via the therapeutic input at a therapeutic rate. The CSF rate is different than the therapeutic rate and/or may be greater than the therapeutic rate. Alternative methods may control a bolus drug infusion to localize the application to a target region.

### PRIORITY

This patent application is a continuation application of U.S. patent application Ser. No. 17/489,620, filed Sep. 29, 2021, entitled, “SUBARACHNOID FLUID MANAGEMENT METHOD AND SYSTEM WITH VARYING RATES,” and naming Gianna Riccardi, William Siopes, Jr., Marcie Glicksman, Anthony DePasqua, Kevin Kalish, Joshua Vose, and Rajan Patel as inventors, which claims priority from 1) provisional U.S. patent application No. 63/084,996, filed Sep. 29, 2020, entitled, “SUBARACHNOID FLUID MANAGEMENT SYSTEM,” and naming Gianna Riccardi, William Siopes, Jr., Marcie Glicksman, Anthony DePasqua, Kevin Kalish, and Joshua Vose as inventors, and 2) provisional

U.S. patent application No. 63/117,975, filed Nov. 24, 2020, entitled, “SUBARACHNOID FLUID MANAGEMENT SYSTEM,” and naming Gianna Riccardi, William Siopes, Jr., Marcie Glicksman, Anthony DePasqua, Kevin Kalish, and Joshua Vose as inventors, the disclosures of which (all three above applications), are incorporated herein, in their entireties, by reference.

### GOVERNMENT RIGHTS

None

### FIELD

Illustrative embodiments generally relate to medical devices and methods and, more particularly, illustrative embodiments relate to devices and methods for managing subarachnoid fluid, such as cerebrospinal fluid (“CSF”), and/or drug delivery that may be used to treat neurodegenerative disorders.

### BACKGROUND

When delivering a drug intrathecally, it is difficult to ensure that the delivered dosage reaches the target anatomy (e.g., part of the brain correlating to a specific disease, such as the cortical versus subcortical). It also is difficult to verify the actual dosage delivered to the target anatomy, as well as control, in real time, the concentration of the drug in the fluid surrounding the target anatomy.

### SUMMARY OF VARIOUS EMBODIMENTS

In accordance with one embodiment of the invention, a CSF management method for use with a patient forms a closed loop CSF circuit between two points on the patient's body. The CSF circuit has a therapeutic inlet to receive a therapeutic material (e.g., a drug), and a pump having a pump outlet to direct CSF along the CSF circuit. The method controls the pump to direct CSF from the pump outlet at a CSF rate that is different from the natural flow rate (i.e., the natural CSF flow rate). The therapeutic material is added to the CSF via the therapeutic input at a therapeutic rate. The CSF rate is different than the therapeutic rate and/or may be greater than the therapeutic rate.

The CSF rate may be a constant rate, or a rate that varies over time. The CSF circuit may be configured so that the CSF simultaneously flows at different rates at two different locations of the CSF circuit. Moreover, the CSF circuit preferably accesses one or more CSF-containing compartments within patient anatomy, such as one or more of the lateral ventricles, the lumbar thecal sac, the third ventricle, the fourth ventricle, and the cisterna magna.

The CSF circuit may have a port into the patient (e.g., a Luer activated valve or another valve). In that case, some embodiments of the CSF circuit have a fluid channel (e.g., a catheter) removably coupled with the port and the pump. To improve performance, the fluid channel also may have a flow sensor, a pressure sensor, or both a flow sensor and a pressure sensor. In addition, or alternatively, the fluid channel may have a controller (e.g., an EEPROM) in communication with the pump configured to track the total number of uses of the fluid channel.

In accordance with another embodiment, a CSF management method for use with a patient forms a CSF circuit to control flow of CSF in the body, adds a therapeutic material to the patient's CSF via the CSF circuit, and directs the therapeutic material (e.g., a drug), via the CSF, toward a prescribed portion of the body. Favorably, the method varies the flow of the CSF in the CSF circuit to localize the CSF at the prescribed portion of the body.

To localize, some embodiments may oscillate the flow of CSF within the CSF circuit for a prescribed time and at a prescribed frequency. To that end, the CSF circuit may have a therapeutic delivery pump and a flow control pump. The therapeutic delivery pump may be directly in line with a reservoir of therapeutic material. Some embodiments may vary the CSF flow rate within the CSF circuit at two or more flow rates at two or more different times. As another option, the CSF circuit may produce pulsatile CSF flow.

The CSF circuit preferably is a closed loop channel in communication with the lower abdomen of a human being. As with other embodiments, the CSF circuit may access one or more CSF-containing compartments with patient anatomy, including one or more of the lateral ventricles, the lumbar thecal sac, the third ventricle, the fourth ventricle, and the cisterna magna.

Some embodiments mix, in a mixing chamber, the therapeutic material and the CSF and/or display a control panel interface configured to control one or both of CSF flow rate and an oscillation frequency. The method may track the progression of the therapeutic material as it flows through the CSF circuit. In that case, the method may vary by reducing the CSF flow rate after the therapeutic material contacts the prescribed portion of the body. When imaging the location of the CSF and/or the therapeutic material, the method may localize as a function of the location of the CSF and/or therapeutic material.

As with other embodiments, this embodiment of the CSF circuit may have a port into the patient (e.g., a Luer activated valve or another valve). In that case, some embodiments of the CSF circuit have a fluid channel (e.g., a catheter or a needle) removably coupled with the port and the pump. To improve performance, the fluid channel also may have a flow sensor, a pressure sensor, or both a flow sensor and a pressure sensor. In addition, or alternatively, the fluid channel may have a controller (e.g., using an EEPROM) in communication with the pump configured to track the total number of uses, shelf life, or sterilization date of the fluid channel.

Illustrative embodiments add a bolus of therapeutic material, such as a full dose in less than 60 seconds.

In accordance with other embodiments, a CSF fluid conduit (e.g., a catheter) directs CSF flow to or from a patient having an exterior port in fluid communication with that patient's subarachnoid space. The CSF fluid conduit is compatible with a CSF circuit having a pump for controlling CSF fluid flow. Accordingly, to those ends, the CSF fluid conduit has a body forming a fluid traversing bore. The body, which has first and second ends in fluid communication with the bore, are removably couplable between the exterior port of the patient and the pump. The bore is in fluid communication with both the exterior port and pump when removably coupled therebetween. Additionally, the body is configured to form a closed loop CSF channel when removably coupled between the pump and the interface, and the CSF channel and bore are in fluid communication with the patient's subarachnoid space when the body is removably coupled. The CSF fluid conduit also has a flow sensor configured to detect flow through the bore of the body, a pressure sensor configured to detect pressure within the bore of the body, and a controller having a communication channel with the pump. The controller has a usage meter configured to track use of the CSF fluid conduit.

The first end of the body preferably is configured to removably couple with the exterior port of the patient via a removable coupling, such as a conventional ANSI standard Luer lock or needle. In a corresponding manner, the second end of the body may be configured to removably couple with the pump.

The removable coupling can be direct or indirect. For example, it may be an indirect connection and, as such, the fluid circuit may have at least one additional component between the first end and the exterior port of the patient. The at least one additional component thus is between the second port and the pump. Of course, related embodiments may removably couple by directly removably coupling with the specific component.

To manage use of the conduit, the controller may be configured to produce indicia indicating at least one use of the CSF fluid conduit. Moreover, when the bore is configured to receive a therapeutic material mixed with CSF, the controller may be configured to control fluid flow as a function of the therapeutic material. The flow sensor may be configured to detect a variety of items, such as the rate of fluid flow through the bore and/or the total volume of fluid through the bore. Further, the controller may be configured to permit a maximum time to use the CSF fluid conduit. The conduit also may have a programmable logic element configured to be programmed to sense or control use of the CSF fluid conduit.

Illustrative embodiments of the invention are implemented as a computer program product having a computer usable medium with computer readable program code thereon. The computer readable code may be read and utilized by a computer system in accordance with conventional processes.

### BRIEF DESCRIPTION OF THE DRAWINGS

Those skilled in the art should more fully appreciate advantages of various embodiments of the invention from the following “Description of Illustrative Embodiments,” discussed with reference to the drawings summarized immediately below.

FIG. 1A schematically shows a cerebrospinal fluid circuit that may be used with illustrative embodiments of the invention.

FIG. 1B schematically shows an external catheter configured in accordance with illustrative embodiments.

FIG. 1C shows a high level surgical flow process in accordance with illustrative embodiments of the invention.

FIG. 2 schematically shows a two pump circuit with drug fed into pump through separate fluid line in accordance with illustrative embodiments.

FIG. 3 schematically shows a two pump circuit with drug introduced directly into fluid line.

FIG. 4A schematically shows a flow control valve circuit that may be used with illustrative embodiments.

FIG. 4B schematically shows a syringe pump dosing circuit with a drug introduced directly into the fluid line configured and usable with illustrative embodiments.

FIG. 5 schematically shows a two-pump circuit with a mixing chamber in accordance with illustrative embodiments.

FIG. 6 schematically shows a flow control valve with a mixing chamber in accordance with other embodiments.

FIGS. 7 and 8 schematically show two different user interfaces in accordance with illustrative embodiments.

FIG. 9 shows a process of localizing drug delivery to a target area of the brain in accordance with illustrative embodiments.

FIG. 10 schematically shows directing flow from lumbar to ventricle in accordance with illustrative embodiments.

FIG. 11 schematically shows directing flow from ventricle to lumbar in accordance with illustrative embodiments.

FIG. 12 schematically shows directing flow from lumbar to ventricle with a pulsatile pattern in accordance with illustrative embodiments.

FIGS. 13A and 13B schematically show bidirectional pump circuits that enable flow in two opposite directions (FIG. 13B between right and left ventricles in the brain) in accordance with illustrative embodiments.

FIG. 14 schematically shows another system interface in accordance with illustrative embodiments.

FIG. 15 shows a process of manually programming drug delivery in accordance with illustrative embodiments.

FIGS. 16A, 16B, and 16C detail an example of illustrative embodiments of the invention.

FIGS. 17A, 17B, and 17C detail another example of illustrative embodiments of the invention.

### DESCRIPTION OF ILLUSTRATIVE EMBODIMENTS

In illustrative embodiments, a system controllably applies a therapeutic material, such as a drug (e.g., methotrexate, a chemotherapy and immunosuppressive drug) to a specific anatomical location within the subarachnoid space or other area. The therapeutic material, which also may be referred to herein as a “drug,” may be applied in a single large volume as a bolus, or dosed gradually over a longer time. To that end, the system has a controller or control system that manages distribution of the therapeutic material within a CSF circuit through which cerebrospinal fluid (“CSF”) flows. Specifically, among other things, the controller (or “control system”) manages pumps, valves, catheters, and/or other structure(s) to control fluid flow, flow direction, and frequencies of certain periodic flows of bodily fluids (e.g., CSF), to provide a more localized and efficient therapeutic application to a patient.

Preferred embodiments enable the therapeutic material to penetrate the blood-rain barrier by either selecting appropriate CSF and therapeutic material flow rates, and/or controlling CSF flow to maintain a bolus of the therapeutic material within CSF at/near a desired location in the CSF circuit. Consequently, using various embodiments, medical practitioners can be more comfortable applying the appropriate application of the therapeutic in the patient, while reducing toxicity and, in some cases, reducing the need for larger volumes of the therapeutic. Details of illustrative embodiments are discussed below.

Many neurodegenerative diseases have been tied to the accumulation of biomolecules (e.g., toxic proteins) contained in cerebrospinal fluid (CSF) or other fluids (e.g., interstitial fluid) within the subarachnoid space (SAS) of a mammalian subject. Problematically, these (e.g., toxic) biomolecules may be secreted and then transported by the CSF to other cells in the body, which process may occur over the span of years. For example, dipeptide repeat proteins (DPRs) and/or TDP-43 have been implicated in neuronal death in the pathology of amyotrophic lateral sclerosis (ALS, or Lou Gehrig's disease), Alzheimer disease (AD), frontotemporal degeneration (FTD), Parkinson's disease (PD), Huntington's disease (HD), and progressive supranuclear palsy (PSP), to name just a few. Hence, research has focused primarily on the removal of harmful DPRs. Techniques for removing DPRs and/or TDP-43 have included: shunting CSF from the CSF space, diluting the CSF (e.g., with an artificial fluid), administering a drug into the CSF, conditioning the CSF, and/or manipulating CSF flow.

Recent breakthrough techniques for handling this problem include ameliorating the CSF, and treating a neurological disorder by removing or degrading a specific (toxic) protein.

Amelioration, as used in various embodiments, involves systems and methods for ameliorating a fluid in the subarachnoid space (SAS) (e.g., a cerebrospinal fluid (CSF), an interstitial fluid (ISF), blood, and the like) of a mammalian subject, unless otherwise particularly distinguished (e.g., referred to as solely CSF). Representative systems may be completely or partially implanted within the body of the mammalian subject (discussed below). Within the body, the systems and/or components thereof may also be completely or partially implanted within the SAS and exposed to the exterior via a port 16 (e.g., a medical valve that provides selective access to the interior system components). These systems execute processes that may occur entirely in-vivo, or some steps that occur extracorporeally. Illustrative embodiments ameliorate with a CSF circuit, discussed below.

Amelioration, for the purpose of illustration, may include changing the physical parameters of the fluid, as well as digestion, removal, immobilization, reduction, and/or alteration, to become more acceptable and/or inactivation of certain entities, including: target molecules, proteins, agglomerations, viruses, bacteria, cells, couples, enzymes, antibodies, substances, and/or any combination thereof. For example, in some embodiments and applications, amelioration may refer to removing toxic proteins from or conditioning one or more of the blood, interstitial fluid, or glymph contained therein, or other fluid, as well as the impact that this removal has on treating diseases or conditions that affect various bodily functions, (i.e., improving the clinical condition of the patient). Moreover, amelioration may be performed by any one of: digestion, enzymatic digestion, filtration, size filtration, tangential flow filtering, countercurrent cascade ultrafiltration, centrifugation, separation, magnetic separation (including with nanoparticles and the like), electrophysical separation (performed by means of one or more of enzymes, antibodies, nanobodies, molecular imprinted polymers, ligand-receptor complexes, and other charge and/or bioaffinity interactions), photonic methods (including fluorescence-activated cell sorting (FACS), ultraviolet (UV) sterilization, and/or optical tweezers), photo-acoustical interactions, chemical treatments, thermal methods, and combinations thereof. Advantageously, various embodiments or implementations of the present invention may reduce levels of toxicity and, after reduced, facilitate maintaining the reduced levels over time.

The extent of amelioration, as reflected by the concentration of the target biomolecules, may be detected through a variety of means. These include optical techniques (e.g., Raman, coherent Stokes, and anti-Stokes Raman spectroscopy; surface enhanced Raman spectroscopy; diamond nitrogen vacancy magnetometry; fluorescence correlation spectroscopy; dynamic light scattering; and the like) and use of nanostructures such as carbon nanotubes, enzyme linked immunosorbent assays, surface plasmon resonance, liquid chromatography, mass spectrometry, circular proximity ligation assays, and the like.

Amelioration may include the use of a treatment system (e.g., UV radiation, IR radiation), as well as a substance, whose properties make it suitable for amelioration. Amelioration of CSF or ameliorated CSF-which terms may be used interchangeably herein-refers to a treated volume of CSF in which one or more target compounds have been partially, mostly, or entirely removed. It will be appreciated that the term removed, as used herein, can refer not only to spatially separating, as in taking away, but also effectively removing by sequestering, immobilizing, or transforming the molecule (e.g., by shape change, denaturing, digestion, isomerization, or post-translational modification) to make it less toxic, non-toxic or irrelevant.

The term, “ameliorating agent” generally refers to a material or process capable of ameliorating a fluid, including enzymes, antibodies, or antibody fragments, nucleic acids, receptors, anti-bacterial, anti-viral, anti-DNA/RNA, protein/amino acid, carbohydrate, enzymes, isomerases, compounds with high-low biospecific binding affinity, aptamers, exosomes, ultraviolet light, temperature change, electric field, molecular imprinted polymers, living cells, and the like. Additional details of amelioration are taught by PCT Application No. PCT/US20/27683, filed on Apr. 10, 2020, the disclosure of which is incorporated herein, in its entirety, by reference. In a similar manner, details for further treatments are taught by PCT Application No. PCT/US19/042880, filed Jul. 22, 2019, the disclosure of which is incorporated herein, in its entirety, by reference.

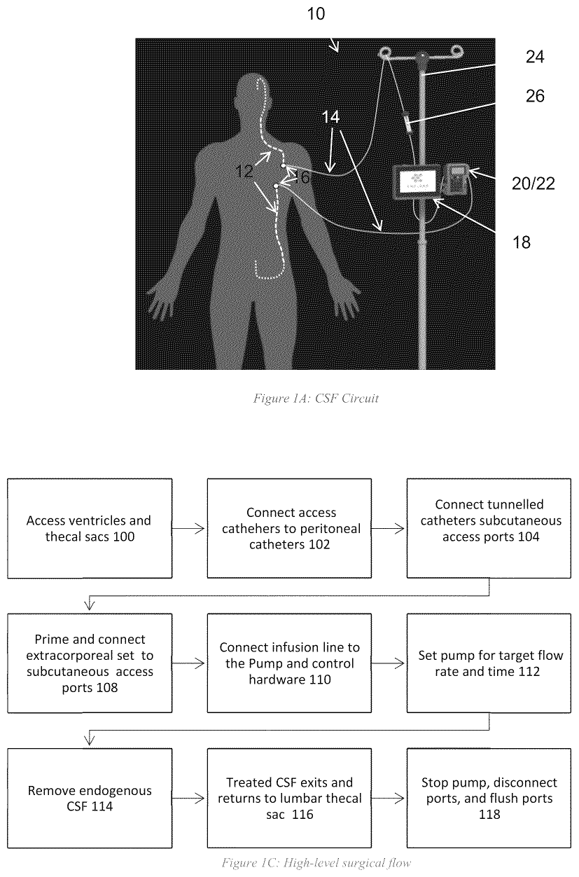

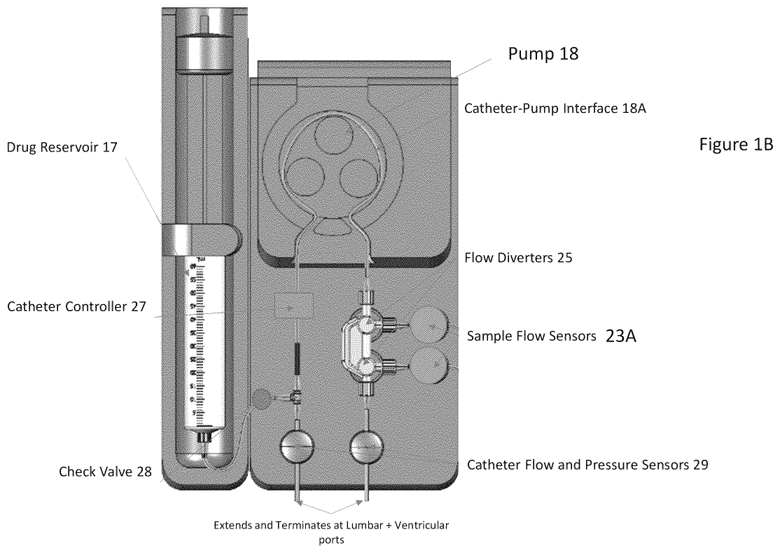

To control CSF flow within the body (e.g., through the ventricle), illustrative embodiments form a CSF circuit/channel (identified by reference number “10”) that manages fluid flow in a closed loop. FIG. 1A, for example, shows one embodiment of such a CSF circuit 10. In this example, internal catheters 12 positioned in-vivo/interior to the body fluidly couple together via the subarachnoid space. To that end, a first internal catheter 12 fluidly couples a prescribed region of the brain (e.g., the ventricle) to a first port 16, which itself is configured and positioned to be accessible by external components. In a corresponding manner, a second catheter couples the lumbar region or the lower abdomen of the subarachnoid space with a second port 16 that, like the first port 16, also is configured to be positioned and accessible by external components. The first and second ports 16 may be those conventionally used for such purposes, such as a valved Luer-lock or removable needle. The first and second internal catheters 12 thus may be considered to form a fluid channel extending from the first port 16, to the ventricle, down the spine/subarachnoid space to the lumbar, and then to the second port 16. These internal components, which may be referred to as “internal CSF circuit components,” are typically surgically implanted by skilled professionals in a hospital setting.

The CSF circuit 10 also has external components (referred to as “external CSF circuit components). To that end, the external CSF circuit components include at least two fluid conduits 14. Specifically, the external CSF circuit components include a first external fluid conduit 14, that couples with the first port 16 for access to the ventricle. The other end of the first external conduit 14 is coupled with a management system 19, which includes one or more CSF pumps (all pumps are generically identified in the figures as reference number “18”), one or more user interface/displays 20, one or more drug pumps 18, and a control system/controller 22. The fluid external fluid conduit 14 may be implemented as a catheter and thus, that term may be used interchangeably with the term “conduit” and be identified by the same reference number 14.

Illustratively, this management system 19 is supported by a conventional support structure (e.g., a hospital pole 24 in FIG. 1A). To close the CSF circuit 10, a second external catheter 14 extends from that same CSF management system 19 and couples with the second port 16 and the management system 19. This management system 19 and external catheters 14 therefore form the exterior part of a closed CSF circuit 10 for circulating the CSF and therapeutic material.

It should be noted that the CSF circuit 10 may have one or more components between the first and second ports 16 and the respective removable connections of the first and second external catheters 14. For example, the first port 16 may have an adapter that couples with the first external catheter 14, or another catheter with a flow sensor may couple between such external catheter 14 and port 16. As such, this still may be considered a removable connection, albeit an indirect fluid connection. There may be corresponding arrangements with the other end of the first external catheter 14, as well as corresponding ends of the second external catheter 14. Accordingly, the connection can be a direct connection or an indirect connection.

The first and second external catheters 12 and 14 preferably are configured to have removable connections/couplings with the management system 19, as well as their respective ports 16. Examples of removable couplings may include a screw-on fit, an interference fit, a snap-fit, or other known removable couplings known in the art. Accordingly, a removable coupling or removable connection does not necessarily require that one forcibly break, cut, or otherwise permanently break the ports 16 for such a connection or disconnection. Some embodiments, however, may enable a disconnection from the first and/or second ports 16 via breaking or otherwise, but the first and/or second ports 16 should remain in-tact to receive another external catheter 14 (e.g., at the end of life of the removed external catheter 14).

FIG. 1B schematically shows more details of the first and/or second external conduits/catheters 14. This figure shows an example of an external catheter 14 operating with other parts of the system. As shown, the system receives a drug reservoir 17 (e.g., a single-use syringe) configured to deliver a dose of therapeutic material (e.g., a drug) that fluidly couples with the catheter 14 via a check valve 28 and T-port 19 on the catheter 14. In addition, the catheter 14 is coupled with a mechanical pump 18 and also preferably includes a sample port 23 with flow diverters 25 for diverting flow toward or away from a sample port 23. The sample port 23 preferably has sample port flow sensors 23A to track samples.

Some embodiments may be implemented as a simple catheter having a body forming a fluid-flow bore with removably couplable ends (or only one removably couplable end). Illustrative embodiments, however, add intelligence to make one or both of these external catheters 14 “smart” catheters, effectively creating a more intelligent flow system. For example, either one or both of the external catheters 14 can have a processor, ASIC, memory, EEPROM (discussed below), FPGAs, RFID, NFC, or other logic (generally identified as reference number “27”) configured to collect, manage, control the device, and store information for the purposes of security, patient monitoring, catheter usage, or communicating with the management system 19 to actively control fluid dynamics of the CSF circuit 10. Among other things, the management system 19 may be configured to coordinate with an EEPROM 27 to control CSF fluid flow as a function of the therapeutic material infusion flow added to the CSF circuit 10 (discussed below) via the check valve 28 at the output of the drug reservoir 17.

As shown in FIG. 1B, one embodiment of the external catheter 14 has the noted electrically erasable programmable read-only memory, EEPROM 27, (or other logic/electronics) that can be implemented to accomplish a variety of functions. Among others, the EEPROM 27 can ensure that the CSF circuit 10 and its operation is customized/individualized to a patient, a treatment type, a specific disease, and/or a therapeutic material. For example, in response to reading information stored in the EEPROM 27, the control system 22 may be configured to control fluid flow as a function of the therapeutic material.

Importantly, as a disposable device, the EEPROM 27 or other logic of the external catheter 14 can be configured to provide alerts, and/or produce or cause production of some indicia (e.g., a message, visual indication, audio indication, etc.) indicating that the external catheter 14 has reached an end of its lifecycle, or indicating how much of its lifecycle remains. For example, an external surface of the catheter 14 may have a tag that turns red when the EEPROM 27 and/or other logic 27 determines that the external catheter 14 has reached its full lifetime use. For example, the external catheter 14 may be considered to have a usage meter, implemented as some logic or EEPROM 27, configured to track use of the CSF fluid conduit 14 to help ensure it is not used beyond its rated lifetime. Moreover, the logic or EEPROM 27 can register with the control system 22 to start use timers to reduce tampering or use beyond a lifetime.

Some embodiments have a printable circuit board (PCB) equipped with a wireless interface (e.g., Bluetooth antenna) or a hardware connection configured to communicate the pump 18 and/or control system 22. The external catheter 14 can be configured to time out after a certain period, capture data, and communicate back and forth with the control system 22 or other off-catheter or on-catheter apparatus to share system specifications and parameters. The intelligent flow catheter 14 can be designed with proprietary connections such that design of knockoffs or cartridges 26 (discussed below) can be prevented to ensure safety and efficacy of the CSF circuit 10 and accompanying processes.

In addition to the management logic, the external catheter(s) 14 also may have a set of one or more flow sensors and/or a set of one or more pressure sensors. Both of those flow sensors are shown generically at reference number 29, and may be located upstream or downstream from their locations in FIG. 1B. For example, the left sensor(s) 29 generically shown in FIG. 1B can be a flow sensor, pressure, or both a flow sensor and pressure. The same can be said for the right sensor(s) 29 generically shown in FIG. 1B. They preferably are positioned between the ports 16 on the body and the remaining components as shown.

Of course, the flow sensor(s) 29 may be configured to detect flow through the bore of the catheter body, while the pressure sensor(s) 29 may be configured to detect pressure within the bore of the body. Among other functions, the flow sensor(s) 29 may monitor flow rate of fluid through the conduit bore and/or total flow volume through the conduit bore. The catheter 14 preferably is configured to have different hardness values at different locations. Specifically, illustrative embodiments may use a mechanical pump 18, as shown and noted above. The pump 18 may periodically urge a compressive force along that portion of the catheter 14 it contacts at its interface 18A with the catheter 14. The outlet of the pump 18 in this case may be the portion of the catheter 14 that is receiving the output of a neighboring compressed catheter portion (e.g., a portion that is adjacent to the compressed catheter portion(s). To operate efficiently, illustrative embodiments form the catheter 14 to have a specially configured hardness at that location (e.g., 25-35 Shore A). Diameter also is important for flow and thus, one skilled in the art should determine appropriate diameters as a function of performance and durometer/hardness. Preferably, the catheter portion that contacts the pump 18 is softer than the remainder of the catheter 14, although both could have the same hardness. Accordingly, the catheter preferably has a variable hardness along its length and may even have a variable diameter.

Alternative embodiments may provide an open-loop CSF fluid circuit 10. For example, the CSF fluid circuit 10 may have an open bath (not shown) to which fluid is added and then removed. The inventors expect the closed-loop embodiment to deliver better results, however, than those of the open-loop CSF fluid circuit 10.

Illustrative embodiments are distributed to healthcare facilities and/or hospitals as one or more kits. For example, one more inclusive kit may include the internal and external catheters 12 and 14. Another exemplary kit may include just the internal catheters 12 and the ports 16 (e.g., for a hospital), while a second kit may have the external catheters 14 and/or a single-use syringe. Other exemplary kits may include the external catheters 14 and other components, such as the management system 19 and/or a CSF treatment cartridge 26. See below for various embodiments of the CSF circuit 10 and exterior components that also may be part of this kit.

Accordingly, when coupled, these pumps 18, valves (discussed below and all valves generally identified by reference number 28), internal and external catheters 14, and other components may be considered to form a fluid conduit/channel that directs CSF to the desired locations in the body. It should be noted that although specific locations and CSF containing compartments are discussed, those skilled in the art should recognize that other compartments can be managed (e.g., the lateral ventricles, the lumbar thecal sac, the third ventricle, the fourth ventricle, and/or the cisterna magna). Rather than accessing the ventricle and the lumbar thecal sac, both lateral ventricles could be accessed with the kit. With both internal catheters 12 implanted, CSF may be circulated between the two lateral ventricles, or a drug could be delivered to both ventricles simultaneously.

In illustrative embodiments, the CSF management system 19 generally manages fluid flow to target anatomy through the CSF circuit 10. To that end, that management system 19 has at least one pump 18 that directs flow of the CSF, and at least one pump 18 that directs flow of a therapeutic material (e.g., a drug) though the CSF circuit 10 to desired anatomy. Alternative embodiments may have more pumps 18 for these functions, or combine pumps 18 for these functions. The management system 19 also has a plurality of valves 28 to control flow, and the control system 22, as noted, is configured to control the pumps 18 to selectively apply the drug-carrying CSF to desired local anatomy. FIG. 1A also shows a user interface 20 that enables a clinician to control drug and fluid parameters in the CSF circuit 10 (discussed below) via the control system 22. Some embodiments may use a monitoring process, such as real-time spectroscopy, to monitor drug concentrations in the CSF. In some of these embodiments, a spectrophotometric sensor may be placed in the CSF circuit 10 to measure the localized concentration of a substance based on its absorption at various wavelengths. For example, some embodiments may use a sensor constructed to measure a single wavelength or multiple wavelengths. The reading taken by the sensor may be relayed to the control system 22, where it would then be stored or processed for various purposes. This signal could be processed for a number of purposes, such as to trigger the control system 22 to alter the fluid flow, flow direction, and/or frequencies of certain periodic flows of bodily fluids (e.g., CSF) to provide a more localized and efficient therapeutic application to a patient in real-time. It will be appreciated that the signal could also be stored or displayed such that the changes to flow, direction or frequencies of period flows could be adjust manually.

FIG. 1C shows a high level surgical flow process that may incorporate the CSF circuit 10 of FIG. 1A in accordance with illustrative embodiments of the invention. It should be noted that this process is substantially simplified from a longer process that normally would be used to complete the surgical flow. Accordingly, this process may have many additional steps that those skilled in the art likely would use. In addition, some of the steps may be performed in a different order than that shown, or at the same time. Those skilled in the art therefore can modify the process as appropriate. Moreover, as noted above and below, many of the materials, devices, and structures noted are but one of a wide variety of different materials and structures that may be used. Those skilled in the art can select the appropriate materials and structures depending upon the application and other constraints. Accordingly, discussion of specific materials, devices, and structures is not intended to limit all embodiments.

The process begins at step 100 by setting up the internal catheters 12 inside the patient. To that end, step 100 accesses the ventricles and thecal sacs using standard catheters and techniques, thus providing access to the CSF. Step 102 then connects access catheters 12 to peritoneal catheters 12, which are tunneled subcutaneously to the lower abdomen. The tunneled catheters 12 then are connected at step 104 to the ports 16 implanted in the abdomen.

At this point, the process sets up an extracorporeal circulation set (i.e., the external catheters 14, or the “smart catheters” in some embodiments). To that end, step 106 may prime and connect the extracorporeal circulation set 14 to the subcutaneous access ports 16. Preferably, this step uses an extracorporeal circulation set, such as one provided by Enclear Therapies, Inc. of Newburyport, MA, and/or the external catheters 14 discussed above. The process continues to step 110, which connects an infusion line or other external catheter 14 to the management system 19, and then sets the target flow rate and time. At this point, setup is complete and treatment may begin (step 112).

The process then removes endogenous CSF from the ventricle. This CSF may then be passed through a digestion region (e.g., through a cartridge 26 having a specific digesting material), where certain target proteins in the CSF are digested. For example, the cartridge 26 may have an inner plenum space 1830 of the cartridge 26 filled with a plurality of (e.g., porous, chromatography resin) beads that have been compression packed. To prevent constituents from entering or escaping from the cartridge 26, a filter membrane may be disposed at the first end of the cartridge 26 and a second filter membrane may be disposed at the second end of the cartridge 26. In some applications, the ameliorating agent may be decorated on the beads 1835.

In some applications, the cartridge 26 may be compression packed with a chromatography resin (e.g., agarose, epoxy methacrylate, amino resin, and the like) that has a protease covalently bonded (i.e. immobilized) to the three-dimensional resin matrix. The selected protease may be configured to degrade and/or removing target toxic biomolecules by way of proteolytic degradation. The resin may be a porous structure having a particle size commonly ranging between 75-300 micrometers and, depending on the specific grade, a pore size commonly ranging between 300-1800 Å. Thus, at a high level, the cartridge 26 has ameliorating agent that removes and/or substantially mitigates the presence of toxic proteins from the CSF.

This and similar embodiments may consider this to be an input for the digesting enzyme. Any location providing access to the drug may be considered to be an input for the drug. At step 116, the treated CSF exits the digestion region and is returned via the CSF circuit 10 to the lumbar thecal sac. The process concludes at step 118, which stops the pump 18 when treatment is complete. The management system 19 then may be disconnected and the ports 16 flushed.

In accordance with illustrative embodiments, the CSF circuit 10 is configured to improve the likelihood of the drug passing through the blood-brain barrier. To that end, the management system 19 enables the user or logic to independently set both the flow rate of CSF circulation (e.g., between 0.05 ml/min and 2.0 ml/min, such as 0.5 ml/min) and the dosing rate of the drug (e.g., between 0.01 ml/min to 2.0 ml/min, such as 0.02 ml/min). Preferably, these rates are different, although they can be the same. In illustrative embodiments, the CSF circulation rate is controlled to be different from the natural CSF flow rate. Note that the natural CSF flow rate is the rate of CSF flow without intervention by outside equipment, such as the pumps 18 and other CSF circuit components—even if it is within a range of typical non-interventionally controlled CSF flow rates. Thus, unless the context dictates otherwise, the non-natural CSF flow rate is the flow rate with such intervention. In other embodiments, the CSF flow rate is simply changed from its truly natural flow rate—i.e., the rate at which the CSF flows without intervention.

Depending on a number of factors, the CSF flow rate may be greater than the rate of drug infusion, while in other embodiments, the CSF flow rate is less than rate of the drug infusion rate. Other embodiments may set them to be equal. Those skilled in the art can select the appropriate flow rate based on a variety of factors, including the drug being delivered, the illness, patient profile, rated pressure of the CSF circuit 10, etc.

The inventors recognized that varying the two rates in a coordinated manner enables more control of the drug dose as well as more control of the drug treatment time. Stated another way, these two independent flow rates enable setting of the dosing rate, which allows the user to optimize drug concentration. At the same time, having the ability to set the flow rate allows the user to control the rate of delivery (as opposed to relying upon natural CSF flow).

As noted in the example below, the inventors were surprised to discover that varying the rates in this manner enabled penetration of the drug across the otherwise difficult to penetrate blood-brain barrier. The selected CSF flow rate may be constant or variable. For example, the CSF flow rate may be set to a first rate for a first period of time, a second rate for a second period of time, and a third rate for a third period of time. As such, various embodiments enable flow of the CSF within the CSF circuit 10 at two or more flow rates at two or more different times. The drug delivery rate may be constant or variable in a similar manner, but coordinated with the CSF flow rate to deliver preferred results.

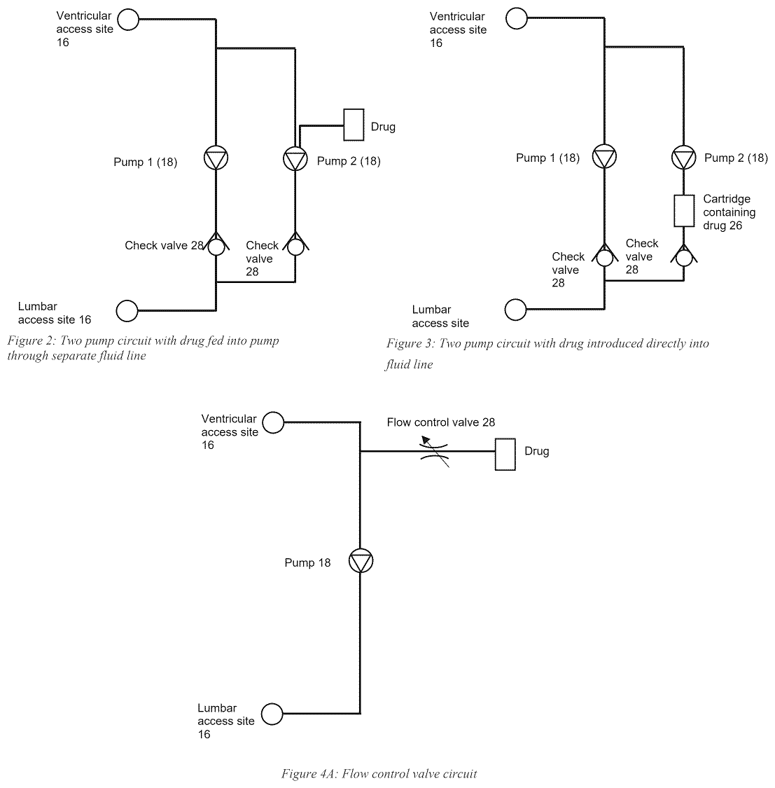

The inventors recognized that a wide variety of different CSF circuit configurations can accomplish the desired goals. FIG. 2 schematically shows a two pump CSF circuit 10 with the drug fed into the pump 18 through a separate fluid line/catheter 12/14 in accordance with illustrative embodiments. In a corresponding manner, FIG. 3 schematically shows a two pump CSF circuit 10 with drug introduced directly into fluid line.

In one embodiment, the CSF circuit 10 has two pumps 18, Pump 1 and Pump 2, to enable a user to set flow rate and dosing rate independently. To that end, Pump 1 may be programmed to control the rate of CSF circulation, while Pump 2 may be programmed to control the dosing rate of the drug to be delivered. Both pumps 18 could be programmed to achieve a desired delivery profile. Check valves 28 or other flow control devices prevent backflow into either pump 18.

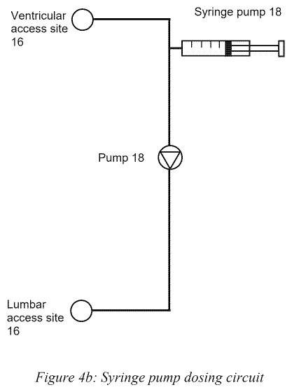

As show, the drug may be fed into the pump 18 through a separate fluid line/catheter (FIG. 2) and input to mix with the patient's CSF in the internal catheter/tubing set 12 before being reintroduced to the body. Alternatively, the drug may also be pre-loaded into a cartridge 26 or other type of drug reservoir and connected directly into the fluid line/catheter 14 (FIG. 3). In this latter embodiment, the CSF mixes with the drug as it flows through the cartridge 26 and tubing set 12/14. FIG. 4A schematically shows another embodiment in which a flow control valve 28 is used in place of Pump 2. In this embodiment, that flow control valve 28 preferably is programmed to control the dosing rate (i.e., the rate of adding the drug to the CSF circuit 10 carrying the CSF.

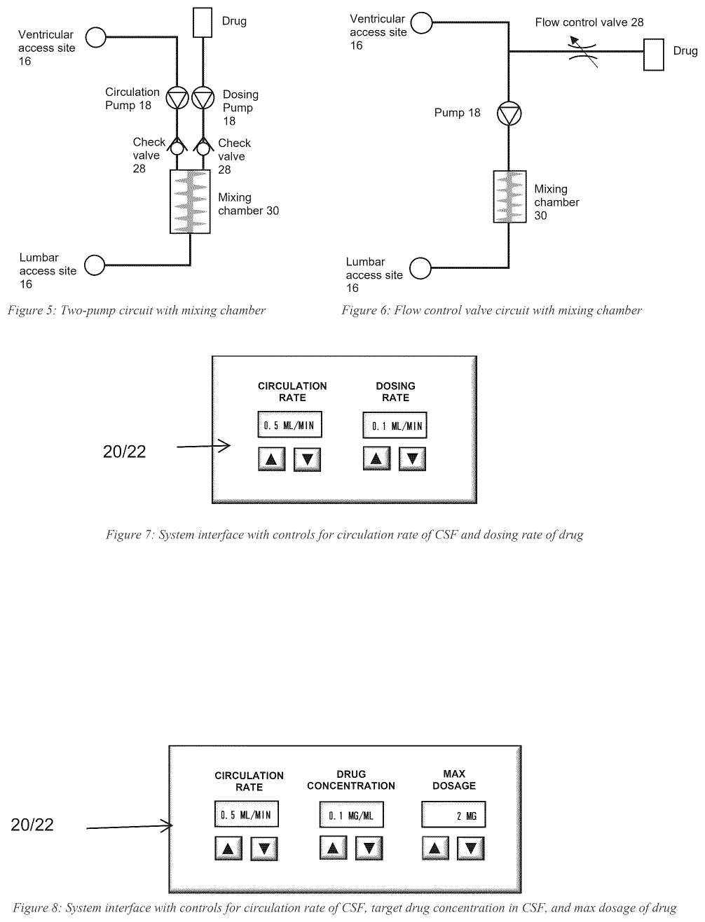

FIG. 5 schematically shows a two-pump circuit 10 with a mixing chamber 30 in accordance with illustrative embodiments. In particular, to ensure a homogeneous mixture of CSF and the drug being delivered, the noted mixing chamber 30 is added to both the two-pump circuit 10 (FIG. 5) and the flow control valve 28 circuit (FIG. 6). The mixing chamber 30 can contain a sensor that provides a readout of a drug's concentration in the CSF, or the management system 19 could simply be programmed to produce a specific drug concentration in the CSF.

In FIG. 5, the CSF-circulating pump 18 and the dosing pump 18 (via an input) feed into the mixing chamber 30 at independent programmable rates. Upon entering the chamber, a small turbine mixes the fluids, and the homogeneous mixture is expelled and returned to the patient anatomy. The same concept applies to FIG. 6, but some in-line mixing occurs before the fluids reach the mixing chamber 30.

Whether controlling dosing rate by a second pump 18 or by a flow control valve 28, CSF delivery may be manually programmed on an interface/display 20 similar to FIGS. 7 and 8. Specifically, FIGS. 7 and 8 schematically show two different user interfaces 20 in accordance with illustrative embodiments. Rather than requiring the user to input a dosing rate, however, the user may specify a drug concentration and the management system 19 responsively may adjust the dosing rate accordingly to achieve that concentration. The user can also input a maximum dosage. After this dosage was reached, the management system 19 would automatically stop treatment. FIG. 8 shows one such interface 20 (e.g., a graphical user interface or a manual interface).

It should be noted that the during actual processing, the CSF flow rate may differ at different parts of the CSF circuit 10—total CSF flow rate in the CSF circuit 10 is not necessarily homogenous. For example, some parts of the CSF circuit 10 may be wider (e.g., certain human geographies) and thus, may be slower than the average CSF circuit flow rate, while other portions may be narrower, causing a nozzle effect and increasing the CSF flow rate at that point. Near the pump 18 (e.g., at the pump outlet), however, the CSF flow rate can be controlled to provide a desired rate across the entire CSF circuit 10, even if that rate may deviate in local parts of the CSF circuit.

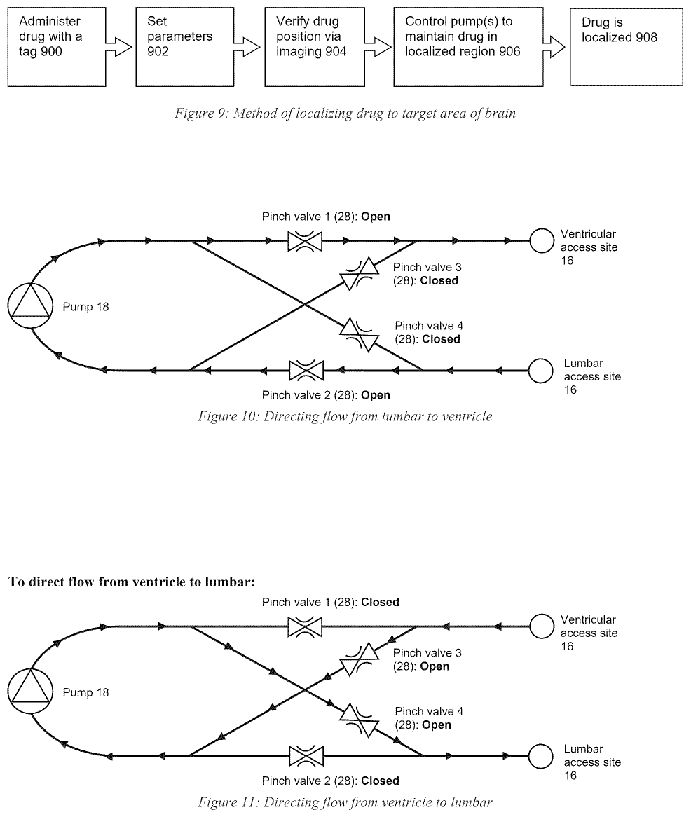

The discussion above relates to delivering a therapeutic material, such as a drug, over a longer infusion period (e.g., 5 minutes, 10 minutes, 30 minutes, 1-6 hours, days, etc.). FIG. 9 shows another embodiment that localizes drug delivery at a target area of the brain using a bolus drug infusion. Specifically, as known by those in the art, a dose of drug can be delivered in a short time period (e.g., 10 seconds, 20 seconds, 60 seconds), or over a longer period (i.e., gradual drug administration, as noted above). The shorter drug delivery is known in the art as a “bolus” drug delivery.

Specifically, to optimize delivery, FIG. 9 alternates the flow direction of the pump 18. The pump 18 thus has programmable controls, via the control system 22, for flow rate and frequency of these alternations. The flow rate and frequency preferably are programmed to achieve a desired delivery profile.

In a manner similar to the other process discussed above, the process of FIG. 9 is substantially simplified from a longer process that normally would be used to complete the localize drug delivery. Accordingly, this process may have many additional steps that those skilled in the art likely would use. In addition, some of the steps may be performed in a different order than that shown, or at the same time. Those skilled in the art therefore can modify the process as appropriate. Moreover, as noted above and below, many of the materials, devices, and structures noted are but one of a wide variety of different materials and structures that may be used. Those skilled in the art can select the appropriate materials and structures depending upon the application and other constraints. Accordingly, discussion of specific materials, devices, and structures is not intended to limit all embodiments.

FIG. 9 therefore delivers a drug intrathecally using positive displacement at a desired flow rate. It may incorporate the components discussed above, as well as principles discussed for other embodiments, such as that discussed above with regard to FIG. 2. The process of FIG. 9 begins at step 900 by adding the drug in a bolus dose to the CSF circuit 10, and/or administering a tag for imaging to the drug. In the latter example, its position can then be tracked using standard imaging techniques to determine when the drug has reached the target anatomy. Alternative embodiments add the drug to the CSF without administering a tag. Such steps may use other techniques to ensure the drug is localized at the desired target anatomy.

Step 902 sets the desired flow rate, direction, timing, and other parameters for the CSF circuit 10 to accomplish the bolus application. For example, specific computer program code on a tangible medium within the control system 22 may cooperate with other components of the CSF circuit 10 to control addition of the therapeutic material, localize the therapeutic material, or both. Next, after step 904 verifies the position of the drug at a target anatomy, step 906 controls the pump 18 to maintain the drug at that target location. Among other ways, step 906 may control the pump(s) 18 to oscillate at a desired flow rate and frequency to contain the drug at that prescribed or desired anatomical location for a pre-set period of time.

After the bolus reaches the target anatomy, the pump 18, which can be programmable and/or have logic, can reverse CSF flow; specifically, the pump 18 can alternate quickly between pushing and pulling flow of the CSF so that the bolus of drug is localized to the target anatomy in the brain (or another target anatomy). In other words, the higher concentration of drug in a portion of the CSF can moved back and forth over the target region. Other embodiments can simply slow down the CSF flow rate to ensure a longer drug application to the target. Either way, these embodiments preferably “soak” the target with the drug, providing a higher quality drug administration. As a result, despite using less of the drug than would be administered by prior art systems, this embodiment still administers a desired amount of the drug to the target by this localizing technique, consequently minimizing toxicity and drug costs (step 908).

It should be noted that “reaching” the target anatomy may be defined by the user or other entity within the control system 22. For example, the portion of CSF in the CSF circuit 10 having the higher concentration of drug (from the bolus) may be considered to have reached the target anatomy when some identifiable portion of it (e.g., the highest concentration, or an interior point within the spread of the drug in the CSF) may be within a prescribed distance upstream of the target, or a prescribed distance downstream of the target. Some embodiments may require the defined portion of CSF with the high drug concentration to actually be at or in contact with that target region. Other embodiments may consider the drug to have “reached” the target simply by calculating the time it should take to reach that area, using artificial intelligence/machine learning, and/or through empirical studies.

Illustrative embodiments can be implemented in a number of different manners with catheters 12/14, pumps 18, valves 28, etc. similar to those discussed above (including the noted external catheters 14). FIGS. 10-14 show several exemplary implementations. In the embodiment shown in FIG. 10, the CSF circuit 10 has four pinch valves 28 on tubing (i.e., external catheters 14), enabling fluid oscillation between opposing flow directions. To flow from lumbar to ventricle (FIG. 10), pinch valves 1 and 2 are opened while pinch valves 3 and 4 are closed. Conversely, to switch flow direction from ventricle to lumbar (FIG. 11), pinch valves 1 and 2 are closed while pinch valves 3 and 4 are opened. Controlling the pinch valves 28 in this manner enables flow direction oscillation. The frequency at which the pinch valves 28 switched between open and closed may be set by the user as could the flow rate of the pump 18 (e.g., via the control system 22). Alternative embodiments may pre-program such parameters into the management system 19.

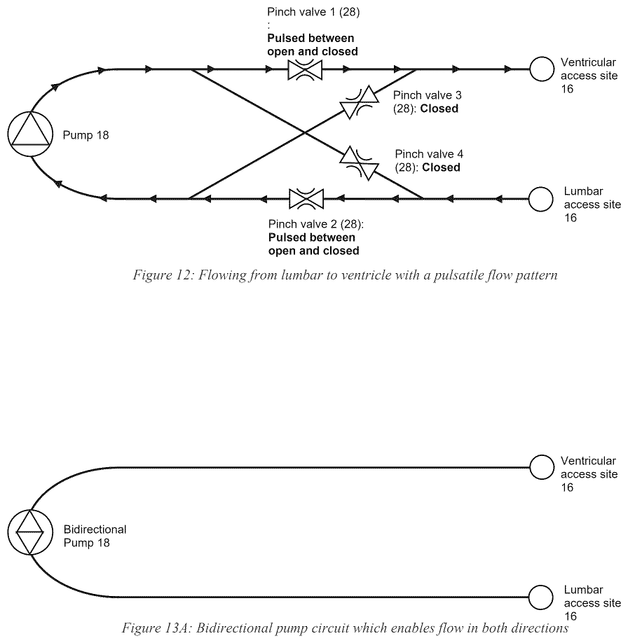

In fact, the same pinch valve 28 configuration (FIG. 12) may be used to create a pulsatile flow pattern. For example, when flowing from lumbar to ventricle, pinch valves 3 and 4 remain closed, while pinch valves 1 and 2 are pulsed (i.e., periodically switched between open and closed) at a frequency set by the user.

The ability to set the frequency at which the pinch valves 28 open and close enables a range of pulsatile effects to be implemented. For example, rather than rapidly switching between open and closed pinch valves 28, the valves 28 can remain closed long enough to build up a set pressure in the fluid line. Shortly after opening the pinch valves 28, a bolus of the drug can be released as a result of the pressure build-up.

Flow direction oscillation and a pulsatile flow pattern could also be produced using a bidirectional pump 18 instead of using pinch valves 28 (e.g., FIG. 13A and FIG. 13B). The pump 18 can be programmed to switch flow directions at a frequency set by the user. While flowing in one direction, the pump 18 can be programmed to pulse by starting and stopping at a frequency also set by the user. Those skilled in the art may use other techniques to provide bidirectional flow.

In addition to those noted above, some embodiments may set the frequency, flow rate, and other parameters as a function of the requirements and structure of the anatomy and devices used in the treatment (e.g., in the CSF circuit 10). Among others, those requirements may include the diameter of the catheters in the CSF circuit 10, physical properties of the drug, the interaction of the drug at the localized region, the properties of the localized region, and other requirements and parameters relevant to the treatment. Those skilled in the art may select appropriate parameters as a function of the requisite properties.

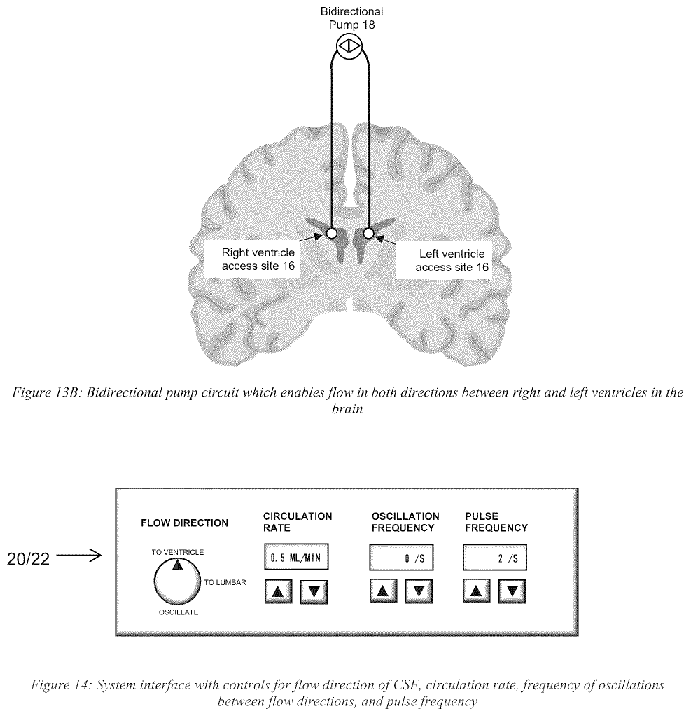

FIG. 14 schematically shows another system interface 20 configured in accordance with illustrative embodiments. Specifically, whether controlling delivery parameters by pinch valve, a bidirectional pump 18, or other means, the delivery profile can be controlled manually with an interface 20, such as the interface 20 shown in FIG. 14, and/or a delivery profile loaded onto the management system 19. As with the other interfaces, this interface 20 may be a fixed control panel, or a graphical user interface on a display device.

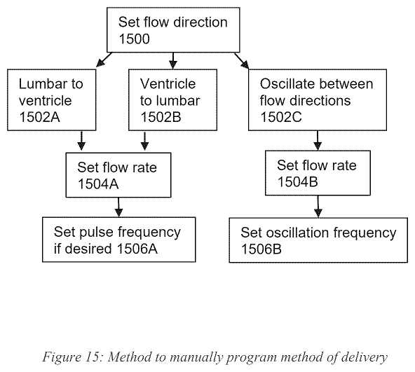

FIG. 15 shows a process of manually programming drug delivery of a bolus in accordance with illustrative embodiments. In a manner similar to the other process discussed above, this process is substantially simplified from a longer process that normally would be used to complete the localize drug delivery. Accordingly, this process may have many additional steps that those skilled in the art likely would use. In addition, some of the steps may be performed in a different order than that shown, or at the same time. Those skilled in the art therefore can modify the process as appropriate. Moreover, as noted above and below, many of the materials, devices, and structures noted are but one of a wide variety of different materials and structures that may be used. Those skilled in the art can select the appropriate materials and structures depending upon the application and other constraints. Accordingly, discussion of specific materials, devices, and structures is not intended to limit all embodiments.

The process begins at step 1500, which sets the flow direction. Three options include lumbar to ventricle (1502A), ventricle to lumbar (step 1502B), or oscillating between flow directions (step 1502C). Next, the process sets the flow rate at steps 1504A or 1504B and sets the frequency of the pulse (step 1506A) or oscillation frequency (step 1506B). Alternative embodiments can be programmed using artificial intelligence algorithms or other program logic.

### Example 1: Administration of Methotrexate to a Sheep Using Illustrative Embodiments

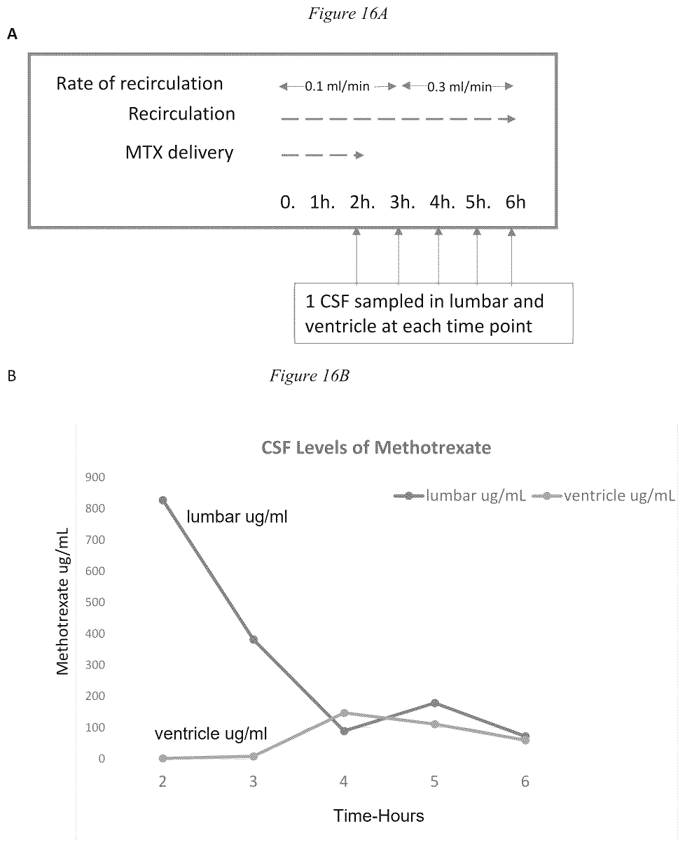

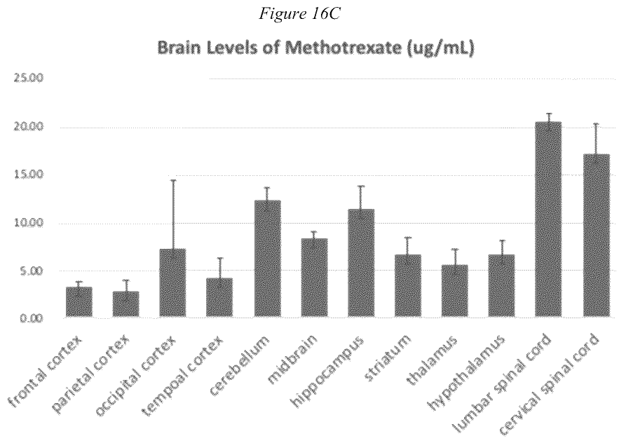

The inventors administered methotrexate to sheep using illustrative embodiments. An outline of the study is depicted by FIGS. 16A-16C. A sheep used for this experiment received the CSF circuit 10 of illustrative embodiments. Circulation was started at the same time that methotrexate was infused. Methotrexate was infused at a gradual rate of 2mLs over 2 minutes and then recirculation from lumbar to ventricle was started at a rate of 0.2 mLs/min. Circulation continued after drug infusion was stopped for four more hours. At zero to three hours, the circulation rate was at 0.1 mL/min and from four to six hours, circulation was at 0.3 mL/min. The dose of methotrexate infused was 12 mg. Drug concentration was measured with LC/MS (liquid chromatography with the mass analysis capabilities of mass spectrometry) in the CSF, spinal cord, and multiple brain regions.

FIG. 16B schematically shows the results. CSF levels of methotrexate were analyzed over time. Drug levels were found to be very high in the lumbar region where the drug was infused and a decline over time was measured except for an increase at 5 hours and then a subsequent decline. CSF levels of methotrexate were very low in the ventricular samples initially, but with time, increased at 4 and 5 hours, before declining to a similar level as the lumbar samples.

FIG. 16C shows how samples from twelve regions of the brain were homogenized and analyzed for methotrexate levels and measured as ug/mL/g of tissue. The x-axis of this graph is drug levels. All areas of the brain and spinal cord had good levels of methotrexate. It will be appreciated that methotrexate that is administered typically subdural in the thigh typically does not cross the blood-brain barrier and would not be found in appreciable levels in brain and spinal cord as a result.

### Example 2: Administration of Antisense Oligonucleotide to a Sheep Using Illustrative Embodiments

The inventors administered an antisense oligonucleotide (ASO) for Huntington's disease to sheep using illustrative embodiments. An outline of the study is depicted in

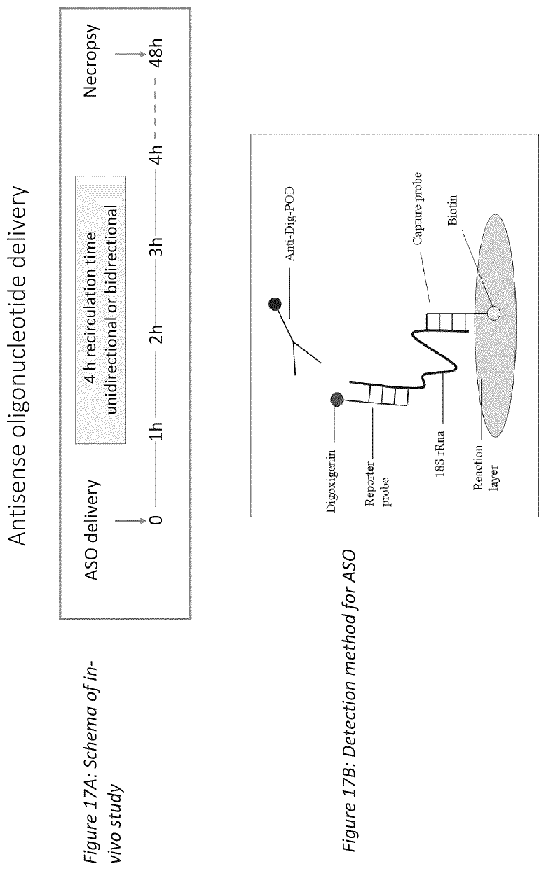

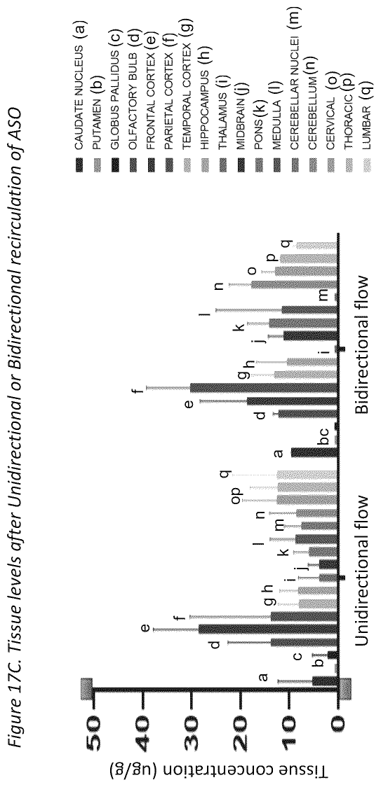

FIGS. 17A-C. Each sheep used for this experiment received the CSF circuit 10 of illustrative embodiments. Circulation was started at the same time that ASO was infused. ASO was infused at a rate of 2 mLs over 2 min. The dose of ASO infused was 30 mg. Circulation continued after drug infusion was stopped for four more hours. At zero to four hours, the circulation rate was at 0.2 mL/min. For unidirectional flow, the direction of the flow was lumbar to ventricle me for the entire 4 h. For the bidirectional flow, the first 1 h of flow was lumbar to ventricle, then the direction of flow was reversed to ventricle to lumbar for 10 min, then switched repeatedly for 10 min intervals for the remainder of the 4 h. All sheep in the study were necropsied after 48 h and tissues were collected to assay drug levels.

The assay for detection of the ASO is depicted in FIG. 17B. Sandwich hybridization ELISA (Enzyme-Linked-immuno-absorption-Assay) quantification used to measure the concentration of CAG repeats in tissue, CSF, and blood samples. Probes comprised of a sequence complementary to the analyte. Capture DNA probe conjugated to biotin label and applied to a streptavidin-coated microtiter plate. Detection DNA probe with digoxigenin label was used. To detect digoxigenin label, anti-digoxigenin (anti-Dig) peroxidase (POD) is reacted with substrate TMB for the signal measurement by an absorption change with a plate reader.

FIG. 17C schematically shows the results. Samples from seventeen regions of the brain were homogenized and analyzed for ASO levels and measured as ug/g of tissue. The x-axis of this graph is drug levels. Most areas of the brain and spinal cord had good levels of ASO. It will be appreciated that the ASO if administered orally, subdurally, or intravenously typically does not cross the blood-brain barrier and would not be found in appreciable levels in brain and spinal cord as a result.

Of course, those skilled in the art should recognize that the above examples are two of many examples that may be used with illustrative embodiments.

Accordingly, illustrative embodiments enable a clinician to more effectively treat various diseases by targeting drug delivery via CSF in the subarachnoid space.

Various embodiments of the invention may be implemented at least in part in any conventional computer programming language. For example, some embodiments may be implemented in a procedural programming language (e.g., “C”), or in an object oriented programming language (e.g., “C++”). Other embodiments of the invention may be implemented as a pre-configured, stand-along hardware element and/or as preprogrammed hardware elements (e.g., application specific integrated circuits, FPGAs, and digital signal processors), or other related components.

In an alternative embodiment, the disclosed apparatus, and methods (e.g., see the various flow charts described above) may be implemented as a computer program product for use with a computer system. Such implementation may include a series of computer instructions fixed either on a tangible, non-transitory medium, such as a computer readable medium (e.g., a diskette, CD-ROM, ROM, or fixed disk). The series of computer instructions can embody all or part of the functionality previously described herein with respect to the system.

Those skilled in the art should appreciate that such computer instructions can be written in a number of programming languages for use with many computer architectures or operating systems. Furthermore, such instructions may be stored in any memory device, such as semiconductor, magnetic, optical, or other memory devices, and may be transmitted using any communications technology, such as optical, infrared, microwave, or other transmission technologies.

Among other ways, such a computer program product may be distributed as a removable medium with accompanying printed or electronic documentation (e.g., shrink wrapped software), preloaded with a computer system (e.g., on system ROM or fixed disk), or distributed from a server or electronic bulletin board over the network (e.g., the Internet or World Wide Web). In fact, some embodiments may be implemented in a software-as-a-service model (“SAAS”) or cloud computing model. Of course, some embodiments of the invention may be implemented as a combination of both software (e.g., a computer program product) and hardware. Still other embodiments of the invention are implemented as entirely hardware, or entirely software.

The embodiments of the invention described above are intended to be merely exemplary; numerous variations and modifications will be apparent to those skilled in the art. Such variations and modifications are intended to be within the scope of the present invention as defined by any of the appended claims.

What is claimed is:

1. A CSF management system for use with a patient having a body with CSF having a natural flow rate, the system comprising:
a catheter configured to at least in part form a CSF circuit between two points on the body;
a therapeutic inlet to the CSF circuit configured to receive a therapeutic material at a therapeutic rate; and
a controller configured to control movement of the CSF at a CSF rate along the CSF circuit, the CSF rate being different from the natural flow rate, the controller configured to set the CSF rate to be different from the therapeutic rate, the CSF rate individually controllable separate from the therapeutic rate.

2. The system as defined by claim 1 wherein the CSF rate is a constant rate.

3. The system as defined by claim 1 wherein the CSF rate varies over time.

4. The system as defined by claim 1 wherein the CSF circuit is configured so that the CSF simultaneously flows at different rates at two different locations of the CSF circuit.

5. The system as defined by claim 1 wherein the CSF circuit is configured to access one or more CSF-containing compartments within patient anatomy, including one or more lateral ventricles, a lumbar thecal sac, a third ventricle, a fourth ventricle, and a cisterna magna.

6. The system as defined by claim 1 wherein the CSF rate is greater than the therapeutic rate.

7. The system as defined by claim 1 wherein the two points on the body comprise ports that permit access to an interior of a patient.

8. The system as defined by claim 1 wherein the catheter comprises a flow sensor, a pressure sensor, or both the flow sensor and the pressure sensor.

9. The system as defined by claim 8 wherein the catheter comprises a catheter controller in communication with a pump, the catheter controller being configured to track and limit use of the catheter.

10. The system as defined by claim 1 wherein the therapeutic inlet comprises a syringe or a fluid bag.

11. The system as defined by claim 1 further comprising a sample port in fluid communication with the catheter.

12. The system as defined by claim 1 further comprising a pump configured to pump CSF and therapeutic material through the CSF circuit under the control of the controller.

13. The system as defined by claim 1 further comprising a display having a graphical user interface to display output relating to the patient and receive input for controlling the CSF circuit.

14. A computer program product for use on a computer system for managing a closed loop CSF circuit between two points on a patient's body having CSF with a natural flow rate, the closed loop CSF circuit comprising a therapeutic inlet, the computer program product comprising a tangible, non-transient computer usable medium having computer readable program code thereon, the computer readable program code comprising:
program code for controlling a pump to direct CSF at a CSF rate through the CSF circuit, the CSF rate being different from the natural flow rate;
program code for controlling addition of therapeutic material to the CSF via the therapeutic inlet into the CSF circuit, the therapeutic material being controlled for addition to the CSF circuit at a therapeutic rate;
program code for individually controlling the CSF rate separate from the therapeutic rate; and
program code for controlling the CSF rate to be different than the therapeutic rate.

15. The computer program product as defined by claim 14 wherein the CSF rate is a constant rate.

16. The computer program product as defined by claim 14 wherein the CSF rate varies over time.

17. The computer program product as defined by claim 14 further comprising program code to control flow of the CSF simultaneously at different rates at two different locations of the CSF circuit.

18. The computer program product as defined by claim 14 wherein the CSF rate is greater than the therapeutic rate.

19. The computer program product as defined by claim 14 wherein the two points on the body comprise ports that permit access to the interior of the patient.

20. The computer program product as defined by claim 14 wherein the CSF circuit comprises an external catheter, the computer program product further comprising program code to track and limit use of the catheter.
